(19) United States
(12) Patent Application Publication
(10) Pub. No.: US 2021/0228091 A1
(43) Pub. Date: Jul. 29, 2021

(54) BIO INFORMATION MEASUREMENT DEVICE AND BIO INFORMATION MEASUREMENT METHOD

(71) Applicant: Seung Wan KANG, Seoul (KR)

(72) Inventors: Seung Wan KANG, Seoul (KR); Dae Keung KIM, Seoul (KR)

(73) Assignee: Seung Wan KANG, Seoul (KR)

(21) Appl. No.: 17/213,667

(22) Filed: Mar. 26, 2021

Related U.S. Application Data
(63) Continuation-in-part of application No. 14/655,218, filed on Jun. 24, 2015.

(30) Foreign Application Priority Data
Jan. 17, 2013 (KR) 10-2013-0005213

(51) Int. Cl. A61B 5/0205; A61B 5/374; A61B 5/346; A61B 5/00

(52) U.S. Cl. CPC A61B 5/0205; A61B 5/374; A61B 5/02416; A61B 5/4035; A61B 5/4058; A61B 5/346

(57) ABSTRACT

A bio information measurement device and a bio information measurement method are provided. The bio information measurement device includes: a heart information acquisition unit for acquiring heart information; a brainwave information acquisition unit for acquiring brainwave information; a control unit for acquiring heart-brain synchronization information on the basis of the heart information and the brainwave information; and a display unit for displaying the heart-brain synchronization information. According to the present invention, it is possible to provide a health index reflecting the influence of both the automatic nervous system and the central nervous system.

### CROSS-REFERENCE TO RELATED APPLICATIONS

This application is a continuation-in-part of U.S. patent application Ser. No. 14/655,218, filed on Jun. 24, 2015, which is a National Stage Entry of PCT/KR2014/000507, filed on Jan. 17, 2014, which is based upon and claims the benefit of priority to Korean Patent Application No. 10-2013-0005213, filed on Jan. 17, 2013. The disclosures of the above-listed applications are hereby incorporated by reference herein in their entirety.

### TECHNICAL FIELD

The present invention relates to a bio information measurement device and bio information measurement method simultaneously considering the automatic nervous system and the central nervous system.

### BACKGROUND ART

As modern medical science develops, a lot of efforts have been made to find an index of disease. However, efforts to find an index of health were not relatively large.

As a marker for health condition known up to now, there are SDNN (Standard deviation of normal to normal) extracted from heart rate variability, HF (high frequency power), heart coherence etc. These are indexes mainly for the automatic nervous system and thus have a limitation that they cannot reflect influences of the central nervous system. There is a conventional system and method for checking heath of blood vessels and stress using pulse wave signal analysis. However, such a conventional system cannot reflect the influences of the central nervous system in the index of health.

### SUMMARY

### 1. Technical Problem

Therefore, the present invention has an object of providing a bio information measurement device and bio information measurement method each capable of grasping a health condition simultaneously considering the influences of the automatic nervous system and the central nervous system.

### 2. Solution to the Problem

A bio information measurement device according to an aspect of the present invention may include a heart information acquisition unit for acquiring heart information; a brainwave information acquisition unit for acquiring brainwave information; and a controller for acquiring heart-brain synchronization information on the basis of the heart information and the brainwave information.

The heart information may include at least one of pulse information, PPG (Photoplethysmograph) information and electrocardiograph information.

The pulse information may include at least one of information about a pulse rate, information about an amplitude of pulse waveform, information about a period of pulse waveform, information about a change of the period of pulse waveform, and information about a frequency of pulse waveform.

The brainwave information may include at least one of information about an amplitude of brainwave waveform and information about a frequency of brainwave waveform.

The controller may acquire the heart-brain synchronization information on the basis of the heart information and the brainwave information. Specifically, the controller may acquire a heart coherence on the basis of the heart information, and acquire a brainwave coherence among a plurality of brainwave waveforms, a phase relation among a plurality of brainwave waveforms, a degree of symmetry among a plurality of brainwave waveform, or a power of brainwave on the basis of the brainwave information.

Furthermore, the controller may acquire the heart-brain synchronization information on the basis of correlation between the heart coherence and the brainwave coherence, correlation between the heart coherence and the phase relation, correlation between the heart coherence and the degree of symmetry or correlation between the heart coherence and the power.

The heart-brain synchronization information may be represented by a degree of proximity to linear function model on the basis of the heart coherence and the brainwave coherence, on the heart coherence and the phase relation, on the heart coherence and the degree of symmetry or on the heart coherence and the power.

The bio information measurement device may further include at least one of an output unit for outputting the heart-brain synchronization information, an interface unit for transmitting the heart-brain synchronization information to external devices, a communication unit for connection to a network, a memory, a user input unit and a camera.

A bio information measurement method according to an aspect of the present invention may include a step of acquiring heart information; a step of acquiring brainwave information; and a step of acquiring heart-brain synchronization information on the basis of the heart information and the brainwave information.

The bio information measurement method may further include a step of outputting the heart-brain synchronization information.

The bio information measurement method may further include a step of transmitting the heart-brain synchronization information to a separate device.

### 3. Effects of the Invention

The present invention can provide an index which allows grasping of a health condition simultaneously considering the influences of the automatic nervous system and the central nervous system.

### BRIEF DESCRIPTION OF THE DRAWINGS

FIG. 1 is a block diagram showing a bio information measurement device, according to some embodiments of the present disclosure;

FIG. 2 is a flowchart showing a method for measuring heart-brain synchronization information, according to some embodiments of the present disclosure;

FIG. 3 is a flowchart showing a particular method for measuring the heart-brain synchronization information, according to some embodiments of the present disclosure;

FIG. 4 shows a change of pulse interval over time, according to some embodiments of the present disclosure;

FIGS. 5A-5B show brainwave waveforms over time, according to some embodiments of the present disclosure;

FIGS. 6A-6C are views showing examples of the heart-brain synchronization information, according to some embodiments of the present disclosure;

FIGS. 7A-7C are views showing examples of method for displaying a heart coherence, a brainwave coherence, and the heart-brain synchronization information, according to some embodiments of the present disclosure; and

FIG. 8 shows a graph that expresses heart-brain synchronization information, according to some embodiments of the present disclosure.

### DETAILED DESCRIPTION

It should be noted that technical terms used in the present specification are used to only describe a specific embodiment and are not intended to limit the present invention. Furthermore, the technical terms used in the present specification should be interpreted as the meaning commonly understood by those skilled in the art unless specifically defined as different meaning in the present specification, and should not be interpreted as excessively comprehensive or excessively narrowed meaning. Furthermore, if the technical terms used in the present specification are wrong technical terms which cannot correctly express concepts of the present invention, they should be understood by being substituted with technical terms which can be correctly understood by the those skilled in the art. In addition, common terms used in the present invention should be interpreted as the meaning defined in a dictionary or from front and back contexts and also should not be interpreted as excessively narrowed meaning.

Furthermore, expressions in the singular form used in the present specification include an expression in the plural form unless otherwise stated explicitly in the context. In the present application, terms such as “comprise (or include)”, “have” etc. and derivatives thereof should not be interpreted as necessarily including all of various elements or steps described in the specification, and thus it should be understood that the terms may not include some of the elements or steps or may include additional elements or steps.

Furthermore, suffixes “module” and “part” for the elements used in the present specification are assigned or used only for the sake of easiness in making the specification, and each suffix itself does not have meaning or role distinct from the relevant element.

Terms including an ordinal number such as “a first^{˜}”, “a second^{˜}” or the like used in the present specification may be used for describing various elements, but the elements should not be limited by the terms. The terms are used only for the purpose of distinguishing one element from another element. For example, a first element may be named a second element and similarly, a second element may be named a first element, without departing from the scope of claims of the present invention.

Furthermore, a term “index” used in the present specification may mean secondary data calculated from data obtained by primarily measuring a bio signal.

Furthermore, a term “a plurality of brainwaves” may mean brainwaves measured at different positions on a scalp in the same time zone or may mean brainwaves measured at the same position on the scalp in different time zones.

Furthermore, instead of mentioning “a brainwave coherence among a plurality of brainwave waveforms, a phase relation among a plurality of brainwave waveforms, a degree of symmetry among a plurality of brainwave waveform, and a power of brainwave”, an expression “brainwave coherence etc.” may be simply used in the present specification.

In the following, preferred embodiments of the present invention will be described in detail with reference to the attached drawings. Regardless of drawing signs, the same or like elements are designated by the same reference numerals, and repeated description thereof will be omitted.

Furthermore, if detailed description of a related known art is thought to obscure the gist of the present invention in the description of the present invention, the detailed description will be omitted. In addition, it should be noted that the attached drawings are only for facilitating understanding of the concept of the present invention and the concept of the present invention should not be construed to be limited by the attached drawings.

A bio information measurement device (100) described in the present specification may include a portable terminal, a stationary terminal, a dedicated terminal for measuring heart-brain synchronization information etc.

Examples of the portable terminal described in the present specification may include a smart phone, a laptop computer, a terminal for digital broadcast, PDA (personal digital assistants), PMP (portable multimedia player), a tablet PC etc. Furthermore, examples of the stationary terminal described in the present specification may include a digital TV, a desktop computer etc.

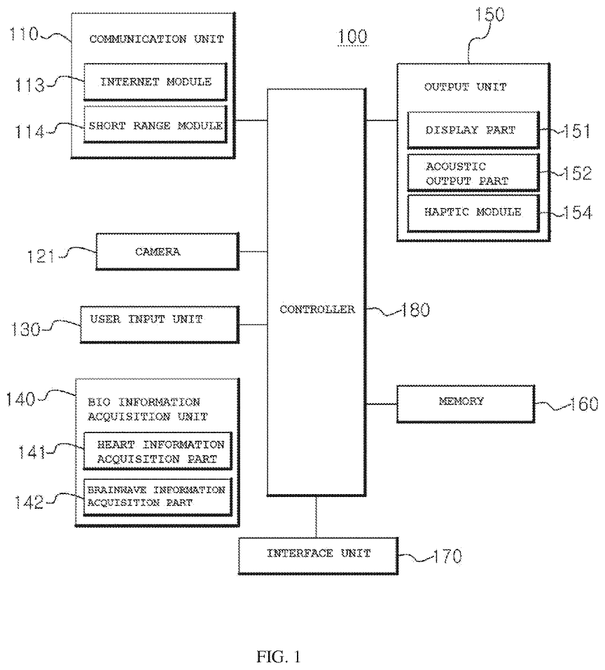

FIG. 1 is a block diagram showing a bio information measurement device (100) according to an embodiment disclosed in the specification.

The bio information measurement device (100) may include a bio information acquisition unit (140) and a controller (180). In addition, the device may include at least one of a communication unit (110), a camera (121), a user input unit (130), an output unit (150), a memory (160) and an interface unit (170), but is not limited to them. The constituent elements illustrated in FIG. 1 are exemplary, and the bio information measurement device having more constituents elements or less constituent elements may be realized.

The communication unit (110) may include one or more module allowing a network communication between the bio information measurement device (100) and a communication system, between the bio information measurement device (100) and a heart information measurer, or between the bio information measurement device (100) and a brainwave measurer.

The communication unit (110) has functions for allowing the bio information measurement device to be connected to the network, and may include one or more module for allowing a communication between the bio information measurement device (100) and the communication system, between the bio information measurement device (100) and an output device, between the bio information measurement device (100) and the heart information measurer, or between the bio information measurement device (100) and the brainwave measurer.

For example, the bio information measurement device (100) may be connected to a separate device such as the output device, the heart information measurer or the brainwave measurer by the communication unit (110). Furthermore, the communication unit (110) may include an internet module (113), short range communication module (114) or the like.

The internet module (113) refers to a module for connection to the internet and may be installed in the bio information measurement device (100) or outside the same. As an internet technology, WLAN (Wireless LAN)(Wi-Fi), Wibro (Wireless broadband), WiMAX (World Interoperability for Microwave Access), HSDPA (High Speed Downlink Packet Access) or the like may be used.

The short range communication module (114) refers to a module for short range communication. As a short range communication technology, Bluetooth, RFID (Radio Frequency Identification), Infrared Data Association (IrDA), UWB (Ultra-Wideband), ZigBee or the like may be used.

The camera (121) processes an image frame such as still image or moving image acquired by an image sensor. The processed image frame may be displayed on a display part (151).

According to an embodiment of the present invention, the camera (121) may be used for acquiring pulse information. For example, the camera (121) captures the moving image of hypodermic blood vessels inside a user's finger, for example an index finger, and the controller (180) may acquire the pulse information on the basis of the captured image.

The user input unit (130) generates input data for control of operation of the bio information measurement device (100) by the user. The user input unit (130) may consist of a key pad, a dome switch, a touch pad (constant voltage/constant current), a jog wheel, a jog switch or the like.

The bio information acquisition unit (140) includes a heart information acquisition part (141) and a brainwave information acquisition part (142).

The heart information refers to information on the activity of heart, and may include, for example, pulse information, PPG (Photoplethysmograph) information and electrocardiograph information.

The pulse is a periodic wave generated when the blood coming out of the heart by heartbeat contacts with a wall of artery. The pulse information may include waveform information about the wave. For example, the pulse information may include information about a pulse rate, information about an amplitude of pulse waveform, information about a period of pulse waveform, information about a change of the period of pulse waveform, information about a frequency of pulse waveform, information about a velocity at which the amplitude of pulse changes or the like.

The photoplethysmograph is an index indicative of contraction and expansion of blood vessels. The photoplethysmograph information may include, for example, information about the contraction and expansion of blood vessels.

The electrocardiograph is a record of the change of electricity locally generated by the activity of heart, and the electrocardiograph information may include information about the change of electricity locally generated by the activity of heart.

The heart information acquisition part (141) may be provided in the bio information measurement device (100). For example, the bio information measurement device (100) may be provided with a pulse measurer for acquiring the pulse information, a PPG (photoplethysmograph) sensor for acquiring the photoplethysmograph information, and an ECG (electrocardiograph) sensor for acquiring the electrocardiograph information. Furthermore, the pulse information may be acquired through the camera (121) and the controller (180), as described above.

Alternatively, the heart information acquisition part (141) may be provided separately from the bio information measurement device (100).

For example, the bio information measurement device (100) may receive the heart information measured by the separate pulse measurer, the PPG sensor and ECG sensor by means of the internet module (113) and the short range communication module (114).

The brainwave is a waveform acquired by recording a current generated according to the activity of brain or by deriving and amplifying the current and recording it. For example, the brainwave information may include information about the amplitude of brainwave waveform, the frequency of brainwave waveform or the like.

The brainwave information acquisition part (142) may be provided in the bio information measurement device (100). For example, the bio information measurement device (100) may be provided with a brainwave measurer.

Alternatively, the brainwave information acquisition part (142) may be provided separately from the bio information measurement device (100).

For example, the bio information measurement device (100) may receive the brainwave information measured by the separate brainwave measurer by means of the internet module (113) and the short range communication module (114).

The output unit (150) is for generating a visual output, an audible output etc. and may include the display part (151), an acoustic output part (152) and a haptic module (154) etc.

The output unit (150) may be provided in the bio information measurement device (100).

Alternatively, the output unit (150) may be provided separately from the bio information measurement device (100).

For example, the bio information measurement device (100) may output information processed in the bio information measurement device through a separate output part by means of the internet module (113) and the short range communication module (114).

The display part (151) displays (outputs) the information processed in the information measurement device (100). For example, the display part may display the heart information or brainwave information acquired by the bio information measurement device. Furthermore, the display part may display a heart coherence, brainwave coherence and heart-brain synchronization information of the user.

The display part (151) may include at least one of LCD (liquid crystal display), TFT LCD (thin film transistor-liquid crystal display, OLED (organic light-emitting diode), flexible display, 3D display, and e-ink display.

The acoustic output module (152) may output an acoustic signal related to the information (for example, the heart information, brainwave information, heart coherence, brainwave coherence, heart-brain synchronization information etc.) processed in the bio information measurement device (100). Such an acoustic output module (152) may include a receiver, a speaker, a buzzer etc.

The haptic module (154) generates various tactile effects which the user can sense. A representative example of the tactile effect generated by the haptic module (154) is vibration. Intensity and pattern etc. of the vibration generated by the haptic module (154) may be controlled. For example, different vibrations may be outputted after being synthesized with one another or may be sequentially outputted.

In addition to the vibration, the haptic module (154) may generate various tactile effects by pin array movable perpendicularly to a contacted skin surface, jetting force or suction force of air through a jetting hole or suction hole, graze against the skin surface, contact of an electrode, effect of stimulation by electrostatic force etc., representation of cold or warmth feel by means of endothermic or exothermic element etc.

The haptic module (154) may be realized so as to deliver the tactile effect through direct contact and to also allow the user to feel the tactile effect through kinesthesia of the user's fingers or arms etc. Two or more haptic modules (154) may be provided according to a constructional aspect of the bio information measurement device.

The haptic module (154) may generate the tactile effect related to the information (for example, the heart information, brainwave information, heart coherence, brainwave coherence, heart-brain synchronization information etc.) processed in the bio information measurement device (100).

The memory (160) may store a program (for example, a method for computing the heart coherence, a method for computing brainwave coherence, and a method for computing the heart-brain synchronization information) for operation of the controller (180) and may temporarily store input/output data (for example, the heart information, brainwave information, heart-brain synchronization information etc.). The memory (160) may include at least one type of storing medium selected from flash memory type, hard disk type, multimedia card micro type, card-type memory (for example, SD or XD memory etc.), RAM (random access memory), SRAM (static random access memory), ROM (read-only memory), EEPROM (electrically erasable programmable read-only memory), PROM (programmable read-only memory), magnetic memory, magnetic disk, and optical disk.

The interface unit (170) may serve as a channel for communication with external devices connected to the bio information measurement device (100). The interface unit (170) receives data or power from the external devices and deliver it to each component of the bio information measurement device (100) or transfers data from the bio information measurement device (100) to the external devices.

The controller (180) usually controls overall operation of the bio information measurement device (100). For example, the controller (180) may acquire the heart coherence on the basis of the heart information. Furthermore, the controller (180) may acquire the brainwave coherence on the basis of the brainwave information. Also, the controller (180) may acquire the heart-brain synchronization information on the basis of the heart coherence and brainwave coherence.

In the following, a particular example of method for measuring the heart-brain synchronization information will be described with reference to the attached drawings.

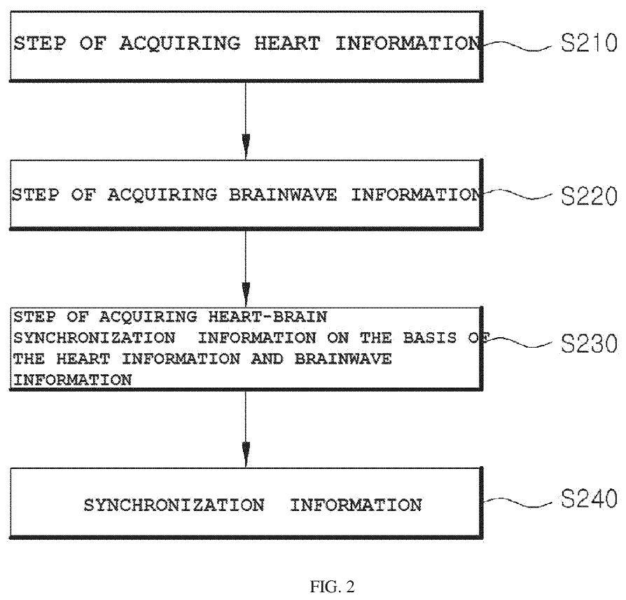

FIG. 2 is a flowchart showing a method for measuring the heart-brain synchronization information according to an embodiment of the present invention.

According to the embodiment of the present invention, the bio information measurement device can acquire the heart information (S210).

In the case where the heart information acquisition part (141) is provided in the bio information measurement device (100), the bio information measurement device (100) can directly acquire the heart information.

Meanwhile, in the case where the heart information acquisition part (141) is provided separately from the bio information measurement device (100), the bio information measurement device (100) may receive the heart information measured by the separate pulse measurer, PPG sensor or ECG sensor by means of the internet module (113), the short range communication module (114) etc.

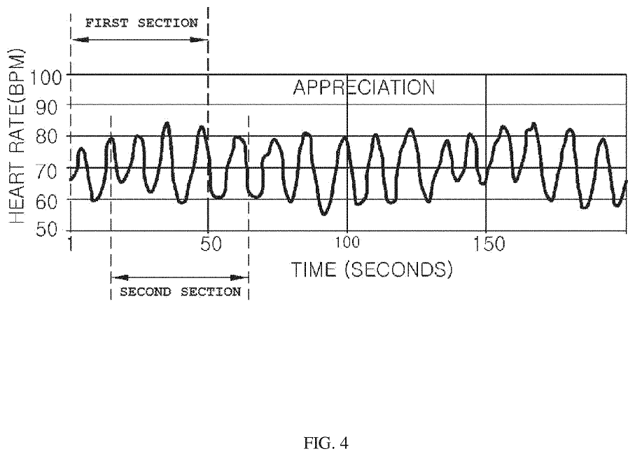

FIG. 4 shows a change of pulse interval over time as data for an example of the heart information.

Referring to FIG. 4, checked can be various pieces of information about the pulse rate, the amplitude of pulse waveform, the period of pulse waveform, the change of period of pulse waveform, the frequency of pulse waveform, and the velocity at which the amplitude of pulse changes etc.

The various pieces of information will be collectively called the pulse information.

According to an embodiment of the present invention, the bio information measurement device can acquire the brainwave information (S220).

In the case where the brainwave information acquisition unit (142) is provided in the bio information measurement device (100), the bio information measurement device (100) can directly acquire the brainwave information.

Meanwhile, in the case where the brainwave information acquisition part (142) is provided separately from the bio information measurement device (100), the bio information measurement device (100) may receive the brainwave information measured by the separate brainwave measurer by means of the internet module (113) and the short range communication module (114).

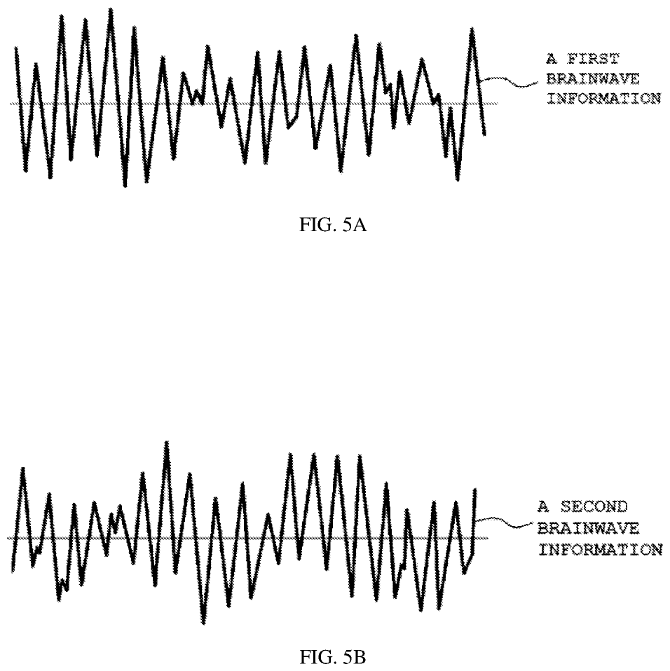

FIG. 5 shows a brainwave waveform over time as data for an example of the brainwave information.

Referring to FIG. 5, the brainwave information may include various pieces of information about the amplitude of brainwave waveform, the frequency thereof etc.

The various pieces of information will be collectively called the brainwave information. According to an embodiment of the present invention, the bio information measurement device can acquire the heart-brain synchronization information on the basis of the heart information and the brainwave information (S230).

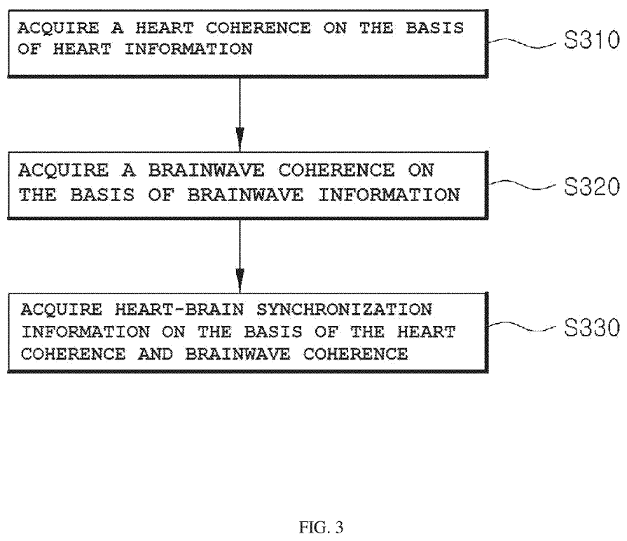

Referring to FIG. 3, a process of acquiring the heart-brain synchronization information will be described in more detail.

FIG. 3 is a flowchart showing a particular method of measuring the heart-brain synchronization information according to an embodiment of the present invention.

According to an embodiment of the present invention, the bio information measurement device can acquire the heart coherence on the basis of the heart information (S310).

The heart coherence is an index indicative of a rate of change of the pulse waveform.

Herein, an example will be described of method for acquiring the heart coherence on the basis of the pulse information.

Referring to FIG. 4, a similarity can be measured between a first waveform in a predetermined section (for example, a first section) and a second waveform in a predetermined section (for example, a second section) which does not completely coincide with the first section.

There are various methods for acquiring the heart coherence.

For example, the controller (180) can transform the first waveform into a frequency domain (a first pulse information) and transform the second waveform into a frequency domain (a second pulse information).

At this time, the controller (180) may determine that the more the frequency band is present in the same range and the higher the similarity between the first pulse information and the second pulse information is, and may determine that the less the frequency band is present in the same range and the lower the similarity between the first pulse information and the second pulse information is. In addition, a maximum likelihood method or cross-correlation method may be used to acquire the heart coherence.

Furthermore, the heart coherence may be acquired by the following Equation 1.

the heart coherence=power of peak center/power of entire band [Equation 1]

Herein, the power of peak center means a power in a predetermined band (for example, 0.03 Hz) taking, as a center, a frequency of which power is the largest in a power spectrum of the pulse information (for example, 0.04˜0.4 Hz).

The predetermined band taking, as a center, a frequency of which power is the largest in the power spectrum may be properly set as necessary. For example, a frequency of the power of peak center may be from 0.08 to 0.15 Hz or from 0.04 to 0.26 Hz.

Furthermore, the power of entire band means an entire power of the power spectrum of the pulse information.

By means of the above equation, the controller (180) can acquire the heart coherence on the basis of the pulse information.

Meanwhile, by applying the way described above, the heart coherence may be acquired on the basis of the PPG (photoplethysmograph) information and electrocardiograph information. For example, the power of entire band and the power of peak band may be acquired on the basis of a frequency spectrum for change of peak interval observed from a time waveform of the PPG information and electrocardiograph information. Then, the heart coherence may be acquired on the basis of the power of entire band and power of peak band thus acquired.

The heart coherence may have a value from 0 to 1.

It can be said that the closer the heart coherence is to 1 and the more regular the change of heart beat over time is.

According to an embodiment of the present invention, the bio information measurement device may acquire the brainwave coherence on the basis of the brainwave information (S320). In addition to the brainwave coherence, a phase relation among a plurality of brainwave waveforms, a degree of symmetry among a plurality of brainwave waveform, or a power of brainwave may be acquired, but only the brainwave coherence is representatively illustrated.

In some embodiments, the power of brainwave is an amplitude of a particular frequency of a brainwave that is measured in real-time.

The brainwave coherence is an index indicative of a degree of coincidence of frequency spectrums for the plurality of brainwaves.

For example, the brainwave coherence may mean a correlation of frequency information among the plurality of brainwave waveforms. The phase relation may be indicative of a phase relation among the plurality of brainwave waveforms, and the degree of symmetry may mean a degree of symmetry among the plurality of brainwave waveforms.

The type of brainwaves generated in a human brain is mainly classified into gamma wave, alpha wave, beta wave, theta wave, delta wave, ILF (Infra Low Fluctuation), and DC depending on the frequency band.

The gamma wave may have a frequency of 30 Hz or more. The alpha wave is a brainwave generated when a body of a person is in a relaxed state while his/her eyes are closed and may have a frequency from 8 Hz to 12 Hz. The beta wave is a predominant brainwave generated when a person stays awake and may have a frequency from 13 Hz to 32 Hz. The theta wave is generated in the state of light sleep, may have a much lower frequency (4 Hz^{˜}8 Hz) than that of the alpha wave and is generated in a boundary state between the consciousness and dream. The delta wave may have a lower frequency (4 Hz or less) than that of the theta wave and is a brainwave most predominantly measured when a person is asleep or is out of consciousness. SCP (Slow cortical potential) may have a frequency of 1 Hz or less.

In the present invention, at least one of the various pieces of brainwave information listed above may be used.

The controller (180) may acquire the brainwave coherence by signal-processing the acquired brainwave. Specifically, the controller (180) amplifies the acquired brainwave, removes unnecessary components from the amplified signal and transforms the signal, from which the unnecessary components have been removed, into a digital signal. Then, the amplified brainwave is Fourier-transformed and an output value for each frequency of the brainwave is computed, whereby the brainwave is analyzed.

According to an embodiment of the present invention, a plurality of pieces of brainwave information may be acquired.

The plurality of pieces of brainwave information may mean a plurality of pieces of brainwave information measured at different positions on a scalp in the same time zone or may mean a plurality of pieces of brainwave information measured at the same position on the scalp in different time zones. Herein, the same time zone means the sameness in start and last times of measurement, and the different time zone means the difference in at least one of the start and last times of measurement.

In a case where the brainwave information acquired at a first position in the same time zone is defined as a first brainwave information, the brainwave information acquired at a second position different from the first position may be defined as a second brainwave information (refer to FIG. 5).

Alternatively, in a case where the brainwave information acquired at the same position in a first time zone is defined as a first brainwave information, the brainwave information acquired in a second time zone different from the first time zone may be defined as a second brainwave information (refer to FIG. 5).

There may be various methods for acquiring the brainwave coherence.

For example, the controller (180) can transform the first brainwave information into a frequency domain and transform the second brainwave information into a frequency domain.

At this time, the controller (180) may determine that the more the frequency band is present in the same range and the higher the similarity between the first brainwave information and the second brainwave information is, and may determine that the less the frequency band is present in the same range and the lower the similarity between the first brainwave information and the second brainwave information is. In addition, a maximum likelihood method or cross-correlation method may be used to acquire the heart coherence.

For example, the brainwave coherence may be acquired by the following Equation 2.

The following Equation 2 can be applied in the case where the brainwave information acquired at a first position in the same time zone is defined as the first brainwave information and the brainwave information acquired at a second position different from the first position is defined as the second brainwave information.

Herein, it is assumed that the brainwave information is acquired at two channels (x, y).

$${Coh}_{x y} = \frac{{\vert CrossPowerSpectrum \vert}^{2}}{PowerSpectrum ( X ) PowerSpectrum ( Y )} \qquad [ Equation 2 ]$$

Herein, |CrossPowerSpectrum| is a density of cross power spectrum between the channel (x) and channel (y), and PowerSpectrum (X) and PowerSpectrum (Y) are densities of auto power spectrum of the channel (x) and channel (y), respectively.

Herein, |CrossPowerSpectrum|² is Cₙ², (aₙuₙ+bₙvₙ)²+(aₙvₙ−bₙuₙ)²=|cospectrum|²+|quadspectrum|². The aₙ and bₙ are Fourier cosine and sine coefficients of the brainwave signal x(t) acquired at the channel (x), respectively. Furthermore, the uₙ and vₙ are Fourier cosine and sine coefficients of the brainwave signal y(t) acquired at the channel (y) different from the channel (x), respectively.

The PowerSpectrum (X) may be expressed by aₙ²+bₙ² and the PowerSpectrum(Y) may be expressed by uₙ²+vₙ².

By the above Equation 2, the controller (180) can acquire the brainwave coherence on the basis of the brainwave information.

The brainwave coherence may have a value from 0 to 1.

The brainwave coherence having a value closer to 1 may mean that frequency spectrums of two brainwaves are similar to or coincide with each other.

The brainwave coherence is a mean of values calculated by the above Equation 2 for a predetermined measurement period of time at all combinations of channels obtainable from a plurality of channels. The brainwave coherence may be measured for each frequency band.

For example, the brainwave coherence is a mean of values calculated by the above Equation 2 for a predetermined measurement period of time at 171 combinations of channels obtainable from 19 channels.

Meanwhile, the phase relation may be acquired by the following Equation 3.

$$Arctan \frac{{\sum }_{N} QuadSpectrum}{{\sum }_{N} CoSpec trum} = Arctan \frac{{\sum }_{N} ( {a}_{n} {v}_{n} - {b}_{n} {u}_{n} )}{{\sum }_{N} ( {a}_{n} {u}_{n} + {b}_{n} {v}_{n} )} \qquad [ Equation 3 ]$$

The phase relation is a mean of values calculated by the above Equation 3 for a predetermined measurement period of time at all combinations of channels obtainable from a plurality of channels. A phase degree may be measured for each frequency band.

For example, the phase degree is a mean of values calculated by the above Equation 3 for a predetermined measurement period of time at 171 combinations of channels obtainable from 19 channels.

Furthermore, the degree of symmetry (amplitude asymmetry) may be acquired by the following Equation 4.

$${\sum }_{N} \frac{\sqrt{( {a}_{n}^{2} + {b}_{n}^{2} )} - \sqrt{( {u}_{n}^{2} + {v}_{n}^{2} )}}{\sqrt{( {a}_{n}^{2} + {b}_{n}^{2} )} + \sqrt{( {u}_{n}^{2} + {v}_{n}^{2} )}} \qquad [ Equation 4 ]$$

The degree of symmetry is a mean of values calculated by the above Equation 4 for a predetermined measurement period of time at all combinations of channels obtainable from a plurality of channels. The degree of symmetry may be measured for each frequency band

For example, the degree of symmetry is a mean of values calculated by the above Equation 4 for a predetermined measurement period of time at 171 combinations of channels obtainable from 19 channels.

Meanwhile, the power is calculated through FFT (Fast Fourier Transformation) (4 seconds Epoch, Hanning Window, Overlapping 50%) after removing noise from the measured brainwave and is a mean value over a predetermined measurement period of time. Furthermore, the power may be calculated at each of a plurality of channels (for example, 19 channels). Meanwhile, the power may be measured for each frequency band (DC, ILF, delta, theta, alpha, beta, gamma etc.)

According to an embodiment of the present invention, the bio information measurement device can acquire the heart-brain synchronization information on the basis of the heart coherence and brainwave coherence (S330).

The heart-brain synchronization information is an index simultaneously indicative of the heart coherence and brainwave coherence in the same time zone.

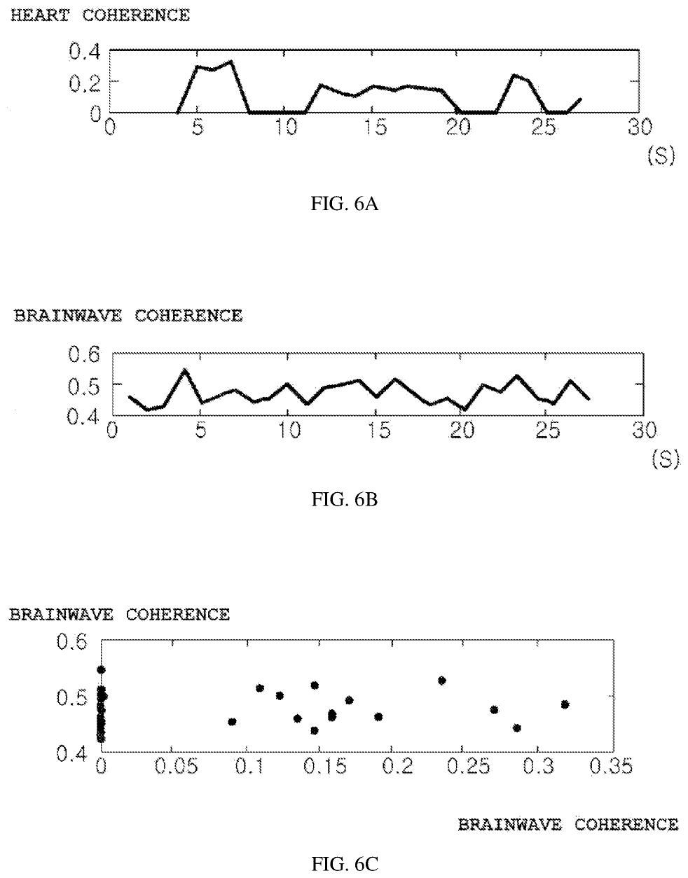

FIGS. 6A-6C are views showing examples of the heart-brain synchronization information.

FIG. 6A is a view showing the heart coherence in a predetermined time section.

FIG. 6B is a view showing the brainwave coherence in a predetermined time section.

FIG. 6C is a view showing the heart-brain synchronization information on the basis of the heart coherence of FIG. 6A and the brainwave coherence of FIG. 6B.

As illustrated in FIG. 6C, a x-axis of the graph designates the heart coherence and a y-axis of the graph designates the brainwave coherence.

That is to say, a correlation between the heart coherence and the brainwave coherence can be grasped through the heart-brain synchronization information.

For example, the correlation may be expressed by a linear function model by means of regression analysis between the heart coherence and the brainwave coherence. Namely, the heart coherence and the brainwave coherence may be expressed by the following Equation 5.

y=a*x+b [Equation 5]

Herein, y is the brainwave coherence and x is the heart coherence. The “a” and “b” may be any rational number satisfying the relational expression.

It may be said that the more a convergence to the regression equation such as the Equation 5 is possible and the larger the heart-brainwave synchronism is, and thus it may be determined a person is in a healthy condition.

Herein, a state where a complete convergence to the regression equation such as the Equation 5 is possible may be defined as a state where a degree of proximity is the highest, and a state where the convergence to the regression equation such as the Equation 5 is not possible may be defined as a state where the degree of proximity is the lowest. Namely, a degree of convergence to the regression equation such as the Equation 5 is defined as the degree of proximity.

According to an embodiment of the present invention, the controller may determine the degree of proximity to the linear function model on the basis of the heart coherence and the brainwave coherence, and the heart-brain synchronism may be grasped through the degree of proximity to the linear function model.

The heart-brain synchronism may be also grasped by determining the correlation between the heart coherence and the phase relation, correlation between the heart coherence and the degree of symmetry or correlation between the heart coherence and the power by application of the same way.

The heart-brain synchronization information may be an index indicative of a health condition simultaneously considering the automatic nervous system and the central nervous system. This is because the heart coherence may be a health index for the automatic nervous system and the brainwave coherence etc. may be a health index for the central nervous system and the heart coherence and brainwave coherence etc. are simultaneously considered for the heart-brain synchronization information.

For example, the controller may grasp the heart-brain synchronization information on the basis of the degree of proximity to thereby determine the health condition.

Meanwhile, it may be determined that the higher the heart coherence is and the better the health condition is. Furthermore, although the heart coherence is high, it may be determined that the person is not in a health condition if the brainwave coherence etc. is not high.

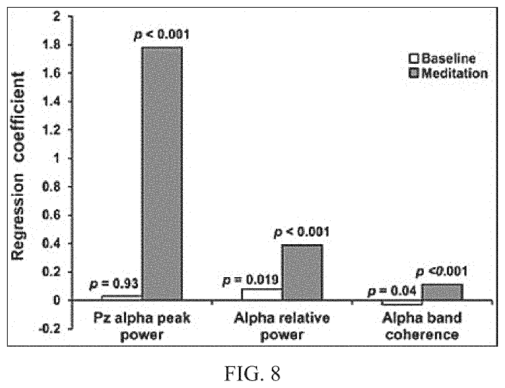

That is to say, the correlation between the heart coherence and the brainwave coherence etc. is grasped and thereby the heart-brain synchronization information is expressed in the form of a graph, based on which the health condition can be determined, as exemplarily shown in FIG. 8.

As shown in FIG. 8, heart coherence, Pz alpha peak power, Alpha relative power, and Alpha band coherence have higher regression coefficients during mediation status than during baseline status. As p value is closer to “0”, there are more data on a regression line. In Equation 5, a and b are more converged, p value is getting lower. Regression coefficients can be determined depending on a characteristic of variable a. A degree of convergence is determined as a degree of proximity. During the mediation status, which can be considered as a mentally healthy status, the degree of proximity is greater than or equal to a preset reference value. During a mentally unhealthy status, the degree of proximity is less than the preset reference value.

By the heart-brain synchronization information, provided can be a health index simultaneously considering the influences of the automatic nervous system and the central nervous system.

Therefore, through the health index, the user may grasp an environment or condition in which the health index is improved.

In more particular embodiments, the controller may perform a linear regression analysis to determine a degree of convergence by using both the heart coherence and the brainwave coherence and by using the above Equation 5, and the controller may determine the degree of convergence as a degree of proximity, determine a healthy condition when the degree of proximity is greater than or equal to a preset reference value, and determine an unhealth condition when the degree of proximity is less than the preset reference value. The preset reference value may be set by the user or by a manufacturer of a bio information measurement device.

Through the process as described above, the controller (180) may acquire the heart-brain synchronization information on the basis of the heart information and the brainwave information (S230).

According to an embodiment of the present invention, the bio information measurement device may output the heart-brain synchronization information (S240).

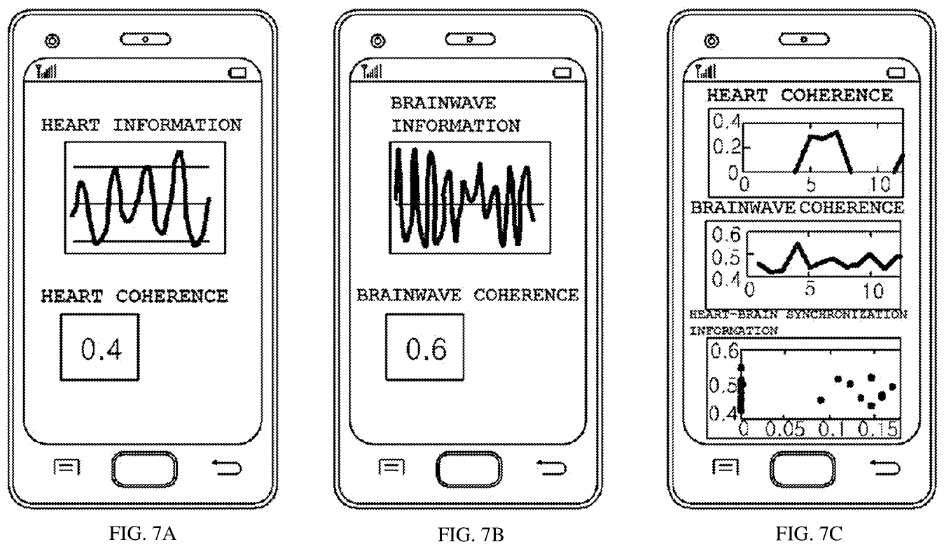

FIGS. 7A-7C are views showing examples of method for displaying the heart coherence, the brainwave coherence, and the heart-brain synchronization information.

As illustrated in FIG. 7A, the display part (151) may display the heart information and the heart coherence.

The heart information and the heart coherence may be continually updated over time and displayed on the display part (151).

Furthermore, the acoustic output module (152) and the haptic module (154) may separately output the heart coherence.

As illustrated in FIG. 7B, the display part (151) may display the brainwave information and the brainwave coherence.

The brainwave information and the brainwave coherence may be continually updated over time and displayed on the display part (151).

Furthermore, the acoustic output module (152) and the haptic module (154) may separately output the brainwave coherence.

As illustrated in FIG. 7C, the display part (151) may display the heart coherence, the brainwave coherence and the heart-brain synchronization information.

Furthermore, the acoustic output module (152) and the haptic module (154) may separately output the heart coherence, the brainwave coherence and the heart-brain synchronization information.

The heart coherence, the brainwave coherence and the heart-brain synchronization information may be continually updated over time and displayed on the display part (151).

Through the information thus outputted, the user may intuitively check the health index simultaneously considering the automatic nervous system and the central nervous system.

As above, by enabling the user to check the heart-brain synchronization information in real time, the user can refer to the information to change his/her own health index in a desirable direction.

### INDUSTRIAL APPLICABILITY

The bio information measurement methods according to the embodiments of the present invention described above may be applied separately from or in combination with each other. Furthermore, steps constituting each embodiment may be applied separately from or in combination with steps constituting another embodiment.

Also, the methods described above may be realized in a recording medium which can be read by a computer or similar device by using software, hardware or combination thereof for example.

According to realization by the hardware, the methods described hitherto may be realized by the use of at least one of ASICs (application specific integrated circuits), DSPs (digital signal processors), DSPDs (digital signal processing devices), PLDs (programmable logic devices), FPGAs (field programmable gate arrays), processors, controllers, micro-controllers, microprocessors, and other electric unit for performing functions.

According to realization by the software, procedures and functions described in the present specification may be realized by separate software modules. The software modules may be realized by a software code written by a proper program language. The software code may be stored in a memory and executed by a processor.

Furthermore, although the embodiment of the present invention has been described above in detail, the scope of claims of the present invention is not limited by the embodiment; rather, included within the scope of claims of the present invention are various modified and improved forms made by those skilled in the art using a basic concept of the present invention defined by the following claims.

What is claimed is:

1. A bio information measurement device comprising:
a heart information acquisition unit configured to acquire heart information and including a photoplethysmograph (PPG) sensor or an electrocardiograph (ECG) sensor;
a brainwave information acquisition unit configured to acquire brainwave information and including a brainwave measurer; and
a controller configured to acquire heart-brain synchronization information on a basis of the heart information and the brainwave information,
wherein the controller is further configured to acquire a heart coherence on a basis of the heart information, wherein the heart coherence is an index indicative of a rate of change of a pulse waveform acquired by the heart information acquisition unit and has a value from 0 to 1,
wherein the controller is further configured to acquire on a basis of the brainwave information:
i) a brainwave coherence among a plurality of brainwave waveforms, wherein a brainwave coherence is an index indicative of a degree of coincidence of frequency spectrums of the plurality of brainwave waveforms and has a value from 0 to 1,
ii) a phase relation among a plurality of brainwave waveforms,
iii) a degree of symmetry among a plurality of brainwave waveform, or
iv) a power of brainwave, on a basis of the brainwave information,
wherein the power of brainwave is an amplitude of a particular frequency of a brainwave that is measured in real-time,
wherein the controller is further configured to acquire the heart-brain synchronization information on a basis of a correlation between the heart coherence and the brainwave coherence, a correlation between the heart coherence and the phase relation, or a correlation between the heart coherence and the degree of symmetry, or a correlation between the heart coherence and the power,
wherein the controller is further configured to perform a linear regression analysis to determine a degree of convergence by using both the heart coherence and the brainwave coherence and by using following relational equation 1:
y=a*x+b
where
y represents the brainwave coherence,
x represents the heart coherence, and
a and b are rational numbers which satisfy the relational equation 1, and
wherein the controller is further configured to
determine the degree of convergence as a degree of proximity,
determine a healthy condition when the degree of proximity is greater than or equal to a preset reference value, and
determine an unhealth condition when the degree of proximity is less than the preset reference value.

2. The bio information measurement device according to claim 1, wherein the heart information comprises PPG (Photoplethysmograph) information or electrocardiograph information.

3. The bio information measurement device according to claim 1, wherein the brainwave information comprises at least one of information about an amplitude of brainwave waveform, information about a period of brainwave waveform, and information about a frequency of brainwave waveform.

4. A bio information measurement method comprising:
acquiring heart information;
acquiring brainwave information;
acquiring a heart coherence on a basis of the heart information, wherein the heart coherence is an index indicative of a rate of change of a pulse waveform acquired by the heart information acquisition unit and has a value from 0 to 1;
acquiring, on a basis of the brainwave information, i) a brainwave coherence among a plurality of brainwave waveforms, wherein a brainwave coherence is an index indicative of a degree of coincidence of frequency spectrums of the plurality of brainwave waveforms and has a value from 0 to 1, ii) a phase relation among a plurality of brainwave waveforms, iii) a degree of symmetry among a plurality of brainwave waveform, or iv) a power of brainwave, on a basis of the brainwave information,
wherein the power of brainwave is an amplitude of a particular frequency of a brainwave that is measured in real-time;
acquiring heart-brain synchronization information on a basis of the heart information and the brainwave information, wherein the heart-brain synchronization information is acquired on a basis of a correlation between the heart coherence and the brainwave coherence, a correlation between the heart coherence and the phase relation, a correlation between the heart coherence and the degree of symmetry, or a correlation between the heart coherence and the power;
performing a linear regression analysis to determine a degree of convergence by using both the heart coherence and the brainwave coherence and by using following relational equation 1:
y=a*x+b [relational equation 1]
where
y represents the brainwave coherence,
x represents the heart coherence, and
a and b are rational numbers which satisfy the relational equation 11
determining the degree of convergence as a degree of proximity;
determining a healthy condition when the degree of proximity is greater than or equal to a preset reference value;
determining an unhealth condition when the degree of proximity is less than the preset reference value.
wherein each step of the method is executed by a bio information measurement device including a processor and a memory.

5. The bio information measurement method according to claim 4, further comprising outputting the heart-brain synchronization information.

6. The bio information measurement method according to claim 5, further comprising transmitting the heart-brain synchronization information to a separate device.
